(19) United States
(12) Patent Application Publication
(10) Pub. No.: US 2003/0153881 A1
(43) Pub. Date: Aug. 14, 2003

(54) Medical instrument with tampon-like section

(76) Inventors: Bruno Roche, AU/ZH (CH); Raymond C. Legrand, Blonay (CH); Max R. Laubscher, Reinach (CH)

Correspondence Address: WALTER A. HACKLER, Ph.D., LAW OFFICES, SUITE B, 2372 S.E. BRISTOL, NEWPORT BEACH, CA, 92660-0755, US

(21) Appl. No.: 10/364,231

(22) Filed: Feb. 11, 2003

(30) Foreign Application Priority Data
Feb. 12, 2002 (DE) 102 05 631.5-44

(51) Int. Cl. A61M 31/00

(52) U.S. Cl. 604/278; 606/199; 606/196

(57) ABSTRACT

A medical instrument (1) comprises a tampon-like section (2) for introduction into a body cavity. The section (2) has an inner part (3), a first cuff (4) surrounding the inner part (3) and a second cuff (5) surrounding at least parts of the first cuff (4) which is permeable for application medium. A medical instrument was produced to stop body fluids from discharge, to stop bleeding and administer application media.

The invention concerns a medical instrument with a section for introducing into body cavities to prevent body fluids from discharge, stop bleeding or permit precise use of application media. This may be suitable e.g. in the rectal field, in the region of the nose, in the vaginal region, in the tracheal or esophageal region.

The invention is based on the object of producing a medical instrument to facilitate the above-mentioned applications.

This object is achieved by a medical instrument comprising a tampon-like section for introduction into a body cavity wherein the section comprises an inner part, a first cuff which surrounds the inner part and a second cuff which surrounds at least parts of the first cuff and is permeable for application media.

The first close-fitting or preformed cuff permits anchoring of the tampon-like section in the body cavity and obtains precise stationary location. The second cuff permits precise discharge of an application medium from the second cuff in this region. The inner part has a reinforcing function and ensures that the cuffs close-fitting to the inner part can be easily and safely introduced into the body cavity.

To realize discharge from the second cuff, the invention proposes to produce either the second cuff of a material which is permeable for the application medium or to provide the second cuff with openings for the application medium.

Close-fitting of the tampon-like section to the body cavity is improved when the inner part is produced from an elastic material. The inner part can be compressed e.g. before introduction, be inserted in this form and automatically expand again when introduced, due to its material property (elasticity).

In a preferred embodiment, longitudinal grooves may be formed on the inner part and produce a non-bulky flat close-fitting of the cuffs on the inner part which further facilitates introduction of the tampon-like section.

For anchoring the tampon-like section in the body cavity, it is advantageous when the first cuff can be activated substantially in the region of the body cavity side end of the tampon-like section. A type of abutment may form.

An additional end support can be produced in that the end of the tampon-like section facing away from the body comprises a stopper. This stopper abuts the outer region of the body cavity and prevents the tampon-like section from being drawn into the body cavity.

The drawing schematically shows a preferred embodiment of the invention which is explained in more detail below with reference to the drawings.

FIG. 1 shows a side view of a medical instrument comprising a tampon-like section for introduction into a body cavity;

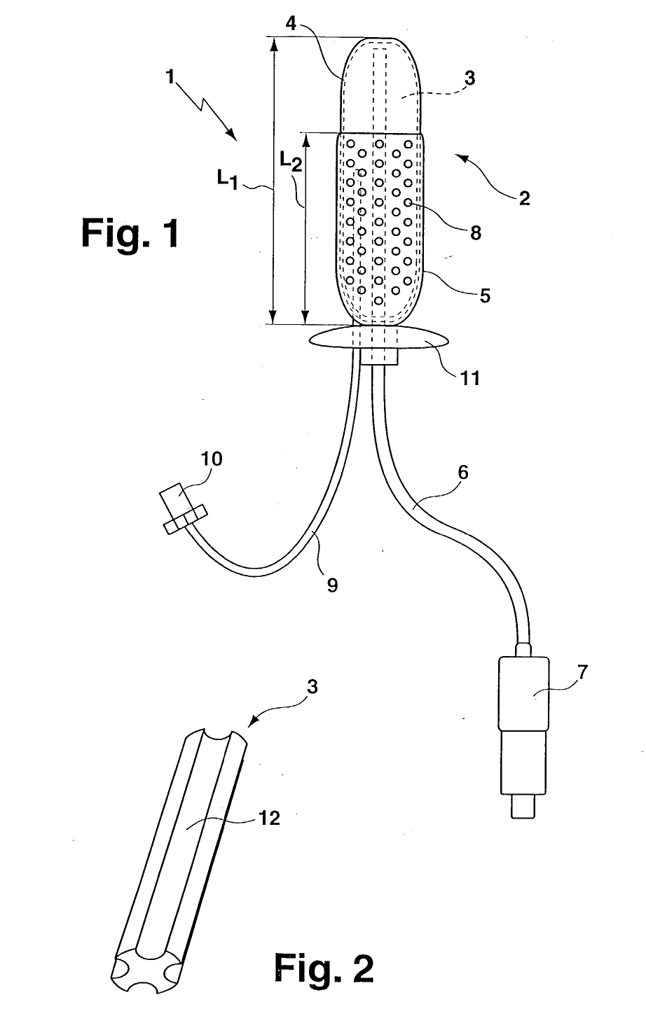

FIG. 2 shows a perspective illustration of an inner part of the tampon-like section of FIG. 1;

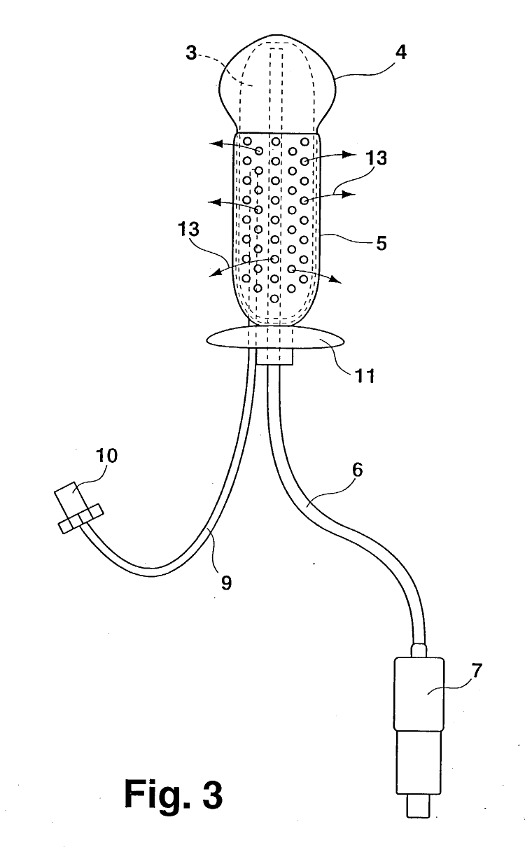

FIG. 3 shows a side view of the medical instrument of FIG. 1 when the cuff of the tampon-like section has been activated.

FIG. 1 shows the design of a medical instrument 1 comprising a tampon-like section 2. The tampon-like section 2 can be inserted into a body cavity and has an inner part 3 formed by a foamed core. The inner part 3 is again coated by a sleeve which is formed by a first 4 and a second cuff 5. The first cuff 4 which can be activated extends over the entire length L₁ of the tampon-like section 2 while the second cuff 5 extends merely over approximately two thirds of this length, i.e. a length L₂, and is disposed above the first cuff 4.

The first cuff 4 can be activated, i.e. filled and pressurized via a feed line 6 and a control balloon 7. A return valve is located within the ventilating line such that the first cuff 4 can be easily evacuated (deactivation) e.g. by means of a syringe or another aid, or be filled (activation).

The second cuff 5 has openings 8 which are shown on an enlarged scale in the drawing. Instead of providing the openings 8, the material of the cuffs 5 may be permeable. The openings 8 serve for applying gases, liquids, fluids or substances (haemostatic solutions, antibiotics, narcotics, analgesic solutions, lubricants and moisturizing solutions etc.) via an application feed line 9. The second cuff 5 is connected with the application feed line 9 and a reservoir which can be connected via the connector 10.

The foamed core 3 or the cuffs 4 and 5 can be drawn onto a hollow cylinder. The cuffs 4 and 5 can be produced from an elastomer, from silicon or another suitable material such as e.g. PVC, and have a round, square, star-shaped, crossed or other contour which prevents slipping out. The contour can adjust to the anatomy of a body cavity into which the tampon-like section 2 with the two cuffs 4 and 5 can be inserted.

A stopper 11 meets the function of an abutment to prevent the introduced tampon-like section 2 from slipping into the body cavity.

In accordance with FIG. 2, the outer contour of the inner part 3 is formed by longitudinal grooves 12 or grooves extending in the longitudinal direction of the inner part 3. This design permits very tight contraction of the cuffs 4, 5 during evacuating since the cuffs 4, 5 can be inserted into the longitudinal grooves 12. As shown in FIG. 1, the evacuated cuffs 4, 5 enlarge the front tampon-like section 2 only slightly compared to the inner part 3.

FIG. 3 shows application of the invention in more detail. This application can refer to the stoppage of bleeding in body openings and application of medication in body openings. This can be suitable e.g. in the rectal region, in the region of the nose, in the vaginal region, in the region of the trachea or the esophagus.

To insert and withdraw the cuffs 4, 5 into and from a body cavity, the cuffs 4, 5 are evacuated and are pressed to the inner part 3. The soft design of the inner part 3 from deformable material permits compression of the inner part 3. On the other hand, the inner part 3 has sufficient rigidity to reinforce the tampon-like part of the medical instrument which facilitates insertion. The radial expansion of the compressed introduced inner part after insertion due to the elasticity (inherent pressure) of the inner part permits stoppage of slight bleeding already. Moreover, medication can be administered via the second cuff 5 as indicated by the flow arrows 13. This could also be sliding agents which facilitate later removal of the tampon-like section.

Regular evacuation which is repeated in intervals prevents gluing of the outer surfaces of the cuff 4, 5 on a wound which is further supported in that the outer surfaces are as smooth as possible.

FIG. 3 shows clearly that the cuff 4 expands through ventilation primarily in the front region of the tampon-like section 2 thereby providing an anchorage in the body cavity in cooperation with the stopper 11 which permits precise application of medication via the openings 8 in the region between the expanded cuff 4 and the stopper 11. The second cuff 5, a type of “excessive balloon” can coat the first cuff 4 either completely or partially and can be filled with solutions. The permeable balloon wall of the cuff 5 permits diffusion of medication. The permeability of the “excessive balloon” 5 can be selected to suit the most different applications and solutions. The “excessive balloon” 5 is provided with the application line 9 which preferably has a Luer lock connection 10 to connect a syringe. The application line 9 can be closed either with a cap or a wedge.

In a preferred application, the shown instrument 1 is used as rectal tampon. The second cuff 5 (“excessive balloon”) covers only two thirds of the first cuff 4 (“balloon”). This produces a pear shape when the first cuff 4 is activated which further prevents sliding out of the rectum.

The inner part may have different foamed material shapes such as round, oval or the crossed shape shown. The different cuff shapes (balloon shapes) are also included in the inventive concept.

### LIST OF REFERENCE NUMERALS

1 medical instrument

2 tampon-like section

3 inner part

4 first cuff

5 second cuff

6 ventilation feed line

7 control balloon

8 opening

9 application feed line

10 connector

11 stopper

12 longitudinal groove

13 flow direction

1. Medical instrument (1) comprising a tampon-like section (2) for introduction into a body cavity, wherein the section (2) has an inner part (3), a first cuff (4) surrounding the inner part (3) and a second cuff (5) surrounding at least parts of the first cuff (4) which is permeable for application media.

2. Medical instrument according to claim 1, characterized in that the second cuff (5) is produced from a material which is permeable for application medium.

3. Medical instrument according to claim 1, characterized in that the second cuff (5) has openings (8) for the application medium.

4. Medical instrument according to claim 1, characterized in that the inner part (3) is produced from an elastic and/or rigid material.

5. Medical instrument according to claim 1, characterized in that the inner part (3) has longitudinal grooves (12).

6. Medical instrument according to claim 1, characterized in that the first cuff (4) can be activated substantially in the region of the body-cavity-side end of the tampon-like section (2).

7. Medical instrument according to claim 1, characterized in that the end of the tampon-like section (2) facing away from the body has a stopper (11).
